SEQUENCE LISTING (6 sequences)

SEQ ID NO: 1 - DNA, 14 bp, Artificial
  PCR Primers
ctgagtgatatccc

SEQ ID NO: 2 - DNA, 20 bp, Artificial
  PCR Primers
cagacagcaaaaaactgtgt

SEQ ID NO: 3 - DNA, 20 bp, Artificial
  PCR Primers
tgtgtcaaaaaacgacagac

SEQ ID NO: 4 - DNA, 18 bp, artificial
  PCR Primers
tagaaggcacagtcgagg

SEQ ID NO: 5 - protein, 9 aa, Homo sapiens
YNIKLWKTF

SEQ ID NO: 6 - protein, 8 aa, Homo sapiens
WKYFTDLF

(19) United States
(12) Patent Application Publication
(10) Pub. No.: US 2005/0202407 A1
(43) Pub. Date: Sep. 15, 2005

(54) Methods and assays for screening anti-neoplastic therapeutic agents

(75) Inventor: Timothy C. Thompson, Houston, TX (US)

Correspondence Address: VINSON & ELKINS L.L.P., 1001 FANNIN STREET, 2300 FIRST CITY TOWER, HOUSTON, TX, 77002-6760, US

(73) Assignee: Baylor College of Medicine, Houston, TX (US)

(21) Appl. No.: 10/998,519

(22) Filed: Nov. 29, 2004

Related U.S. Application Data
(60) Provisional application No. 60/525,986, filed on Dec. 1, 2003.

(51) Int. Cl. C12Q 1/00

(52) U.S. Cl. 435/4

(57) ABSTRACT

The present invention provides methods and assays for screening and identifying agents that will be useful in the treatment and prevention of neoplastic disorders. The methods and assays of the present invention are particularly useful for identifying therapeutic agents for steroid sensitive neoplastic disorders such as prostate cancer.

### STATEMENT REGARDING FEDERALLY SPONSORED RESEARCH OR DEVELOPMENT

This application claims the priority benefit of U.S. Provisional Application Ser. No. 60/525,986 filed Dec. 1, 2003.

The research leading to the present invention was supported, in part, by National Cancer Institute Grants No. RO 168814 and No. PO50/SPORE/58204. Accordingly, the United States Government has certain rights in the invention.

### BACKGROUND OF THE INVENTION

1. Field of the Invention

The present invention relates to methods and assays for screening and identifying agents that will be useful in the treatment and prevention of neoplastic disorders. The methods and assays of the present invention are particularly useful for identifying therapeutic agents for androgen sensitive neoplastic disorders.

2. Description of the Related Art

Cancer cells are typically defined by uncontrolled growth and uncontrolled invasion of normal tissue. Cancerous cells can divide in defiance of the normal growth constraints leading to a localized growth or tumor. Some cancer cells gain the ability to migrate away from the primary tumor site and invade other healthy tissues. A tumor or neoplasm that remains noninvasive or benign may be completely cured by surgically removing the mass. A cancer is referred to as malignant if its cells are capable of invading surrounding non-cancerous tissue and form secondary tumors or metastases. The more widely a tumor metastasizes, the harder it is to eradicate and treat.

Most types of cancer develop in stages from mildly benign into malignant neoplasms. Prostate cancer provides one of the more clear examples of the progression of normal tissue to benign neoplasm to malignant neoplasm. The prostate is an encapsulated organ of the mammalian male urogenital system located at the base of the bladder. The prostate is partitioned into zones referred to as the central, peripheral and transitional zones, all of which surround the urethra. Histologically, the prostate is a highly microvascularized gland comprising fairly large glandular spaces lined with epithelium which, along with the seminal vesicles, supply the majority of fluid to the male ejaculate. As an endocrine-dependent organ, the prostate responds to both the major male hormone, testosterone, and the major female hormones, estrogen and progesterone. Testicular androgen is considered important for prostate growth and development because, in both humans and other animals, castration leads to prostate atrophy and, in most cases, an absence of any incidence of prostatic carcinoma.

In its more aggressive form, malignant transformed prostatic tissues escape from the prostate capsule and metastasize invading locally and throughout the bloodstream and lymphatic system. Metastasis, which are cancerous growths that are discontinuous with the primary tumor, can occur through direct seeding, lymphatic spread and hematogenous spread. All three routes have been found to occur with prostatic carcinoma. It has been estimated that about 60% of newly diagnosed prostate cancer patients will have metastases at the time of initial diagnosis.

Prostate cancer is the most common malignancy in men in the USA, resulting in an estimated 41,800 deaths in 1997. (Parker S L, et al., Cancer J Clin 47: 5-27, 1997). The widespread use of prostate-specific antigen (PSA) has dramatically increased the number of patients diagnosed with prostate cancer and generally lowered the stage of disease at diagnosis. (Scardino P T, Urol. Clin. N. Am. 16:635-655, 1989; Epstein J L, et al., JAMA 271: 368-374, 1994). Nevertheless, 5%-10% of cancers detected by PSA screening are clinically advanced and not candidates for radical prostatectomy. Despite surgical removal of the prostate, 30%-60% of men treated will have recurrence of cancer within 5 years, suggesting that the clinical stage of the patients undergoing surgery was highly inaccurate. 20%-57% of patients undergoing definitive surgery with presumed localized disease will have rising PSA following treatment, also indicative of local or distant residual disease. (Ohori M, et al., J. Urol. 154:1818-1824, 1995; Zeitman A L, et al., Urology 43: 828-833, 1994). Unfortunately, neither of these conditions is amenable to curative therapy.

Surgery or radiotherapy is the treatment of choice for early prostatic neoplasia. Surgery involves complete removal of the entire prostate (radical prostatectomy), and often removal of the surrounding lymph nodes, or lymphadenectomy. Radiotherapy, occasionally used as adjuvant therapy, may be either external or interstitial using ¹²⁵I.

Endocrine therapy is the treatment of choice for more advanced forms. The aim of this therapy is to deprive the prostate cells, and presumably the transformed prostate cells as well, of testosterone. This is accomplished by orchiectomy (castration) or administration of estrogens or synthetic hormones which are agonists of luteinizing hormone-releasing hormone. These cellular messengers directly inhibit testicular and organ synthesis and suppress luteinizing hormone secretion which in turn leads to reduced testosterone secretion by the testes. In normal prostate, removal of androgenic hormones results in regression of the gland involving apoptosis of more than 60% of the luminal epithelial cells. Although often initially sensitive to removal of androgens, prostate cancer cells eventually lose this response and continue to grow and spread even in the absence of androgenic steroids. Despite the advances made in achieving a pharmacologic orchiectomy, the survival rates for those with late stage carcinomas are rather bleak.

Current therapeutic regimens for metastatic disease typically involve both chemical and surgical androgen ablation, which although they demonstrably extend life when compared to untreated patients, almost invariably result in the development of hormone-refractory disease and the demise of the patient. An alternate explanation for the predictable failure of androgen ablation is that a certain population of the prostatatic cancer cells may become sensitized to the minor amounts of androgen that persists after ablation and, therefore, continue to grow. This is possible because although chemical and surgical ablation drastically reduces the quantity of testosterone in the body measurable levels of the hormone persist either as uncleared amounts or as newly synthesized molecules which have been converted to testosterone from other steroid hormones (by the adrenals for example).

With the advent of molecular biology, various investigators in laboratories have attempted to understand the molecular biology of castration-induced regression of the prostate at a more mechanistic level. The model systems selected almost invariably compared mRNAs produced prior to castration and during castration-induced regression using rat prostate model systems in vivo. These model systems yield gene activities that may be involved in castration-induced regression but could also be involved in activities that are not directly relevant or related to castration-induced regression but were stimulated by removal of androgenic steroids. It is anticipated that only a small fraction of gene activities modulated by steroid withdrawal would indeed be involved in castration-induced regression and, therefore, significant confounding background activity would be seen in these existing model systems.

Previously, Yang et al. reported that caveolin-1 (cav-1) levels were elevated in metastatic mouse and human prostate cancer (Yang, G., et al. 1998. Clin. Cancer Res. 4:1873-1880). cav-1 is a major component of caveolae, flask-shaped membrane invaginations which are involved in multiple cellular processes, including the regulation and transportation of cellular cholesterol and lipids, clathrin-independent endocytosis, and signal transduction (Harris, J., et al. 2002. Trends Immuniol. 23:158-164; Ikonen, E., et al. 2000. Traffic 1:212-217; Schroeder, F., et al. 2001. Exp. Biol. Med. (Maywood) 226:873-890; Shaul, P. W., et al. 1998. Am. J. Physiol. 275:L843-L851; Sternberg, P. W., et al. 1999. Nat. Cell. Biol. 1:E35-E37). The participation of cav-1 in these critical pathways involves the interaction of cav-1 with a relatively large number of molecules in either a scaffolding binding-dependent or -independent manner (Lu, M. L., et al. 2001. J. Biol. Chem. 276:13442-13451; Smart, E. J., et al. 1999. Mol. Cell. Biol. 19:7289-7304). The wide spectrum of molecular interactions involving cav-1 is consistent with important, context-dependent roles for cav-1 in signal transduction, molecular transport, and other regulatory activities.

The biological functions of cav-1 in cancer are complex, multifaceted, and somewhat controversial (Massimino, M. L., et al. 2002. Cell. Signal. 14:93-98; Razani, B., et al. 2001. Biochem. Soc. Trans. 29:494-499; Thompson, T. C., et al. 1999. Apoptosis 4:233-237, Thompson, T. C., et al. 2001; Molecular pathways that underlie prostate cancer progression: the role of caveolin-1. In L. Chung (ed.), Prostate cancer in the 21st century. Humana Press, Totowa, N.J.). Numerous experimental results indicate that cav-1 is a growth suppressor gene (Engelman, J. A., et al. 1997. J. Biol. Chem. 272:16374-16381; Galbiati, F., et al. 2001. Mol. Biol. Cell 12:2229-2244; Lee, S. W., et al. 1998. Oncogene 16:1391-1397). Some investigators have asserted that cav-1 is also a tumor suppressor gene (Razani, B., et al. 2001. Biochem. Soc. Trans. 29:494-499). Although there is clear evidence for negative growth regulation in specific cell types, the biological and genetic evidence for a tumor suppressor function for cav-1 is lacking at this time. However, the available data are consistent with a role for negative growth regulation in specific cell lines and lineages (reviewed by Mouraviev et al. [Mouraviev, V., et al. 2002. J. Urol. 168:1589-1596]). Interestingly, there is also substantial evidence that cav-1 is overexpressed in metastatic cells and promotes cell survival in prostate cancer and other malignancies.

Since the first report that elevated expression of cav-1 is associated with prostate and breast cancer in 1998 (Yang, G., et al. 1998. Clin. Cancer Res. 4:1873-1880), this initial observation has been extended in prostate cancer (Gob, J., et al. 2001. Neoplasia 3:331-338; Tso, C. L., et al. 2000. Cancer J. 6:220-233; Wu, D., et al. 2002. Cancer Res. 62:2423-2429; Yang, G., et al. 1999. Cancer Res. 59:5719-5723), and there have been numerous reports of cav-1 overexpression in aggressive stages of other malignacies, including colon cancer (Fine, S. W., et al. 2001. Am. J. Clin. Pathol. 115:719-724), bladder cancer (Rajjayabun, P. H., et al. 2001. Urology 58:811-814), esophageal squamous cell cancer (Hu, Y. C., et al. 2001. Clin Cancer Res. 7:3519-3525; Kato, K., et al. 2002. Cancer 94:929-933), papillary carcinoma of the thyroid (Ito, Y., et al. 2002. Br. J. Cancer 86:912-916), ovarian cancers (Davidson, B., et al. 2001. Gynecol. Oncol. 81:166-171), myeloma (Podar, K., et al. 2003. J. Biol. Chem. 278:5794-5801), pancreatic ductal adenocarcinoma (Suzuoki, M., et al. 2002. Br. J. Cancer 87:1140-1144), and lung cancer (Ho, C. C., et al. 2002. Am. J. Pathol. 161:1647-1656). Overall, an impressive accumulation of data indicates that cav-1 is overexpressed in aggressive forms of specific malignancies and likely contributes to cancer progression.

Recent studies indicate that protein kinase B (PKB)/Akt activities are central to the development and maintenance of specific malignancies (reviewed in references Blume-Jensen, P., et al. 2001. Nature 411: 355-365, Brazil, D. P., et al. 2001. Trends Biochem. Sci. 26:657-664, Nicholson, K. M., et al. 2002. Cell. Signal. 14:381-395, Testa, J. R., et al. 2001. Proc. Natl. Acad. Sci. USA 98:10983-10985, and Vivanco, I., et al. 2002. Nat. Rev. Cancer 2:489-501). Akt is constitutively active in many human cancers due to amplification of the Akt gene or as a result of amplification or mutations in components of the signaling pathway that regulate Akt activities (Nicholson, K. M., et al. 2002. Cell. Signal. 14:381-395, Vivanco, I., et al. 2002. Nat. Rev. Cancer 2:489-501). In healthy cells, the tumor suppressor PTEN functions as a major negative regulator of the phosphatidylinositol 3-kinase (PI3-K)/Akt pathway through dephosphorylation of PI-3,4-P2/PI-3,4,5-P3 (Cantley, L. C., et al. 1999. Proc. Natl. Acad. Sci. USA 96:4240-4245; Di Cristofano, A., et al. 2000. Cell 100:387-390; Leslie, N. R., et al 2002. Cell. Signal. 14:285-295). On the other hand, the phosphorylation state of Akt can also be controlled by serine/threonine protein phosphatases (Cohen, P. T. 2002. J. Cell Sci. 115:241-256; Janssens, V., et al. 2001. Biochem. J. 353:417-439; Millward, T. A., et al. 1999. Trends Biochem. Sci. 24:186-191; Schonthal, A. H. 2001. Cancer Lett. 170:1-13; Wera, S., et al. 1995. Biochem. J. 311:17-29). PP1 and PP2A are two major classes of serine/threonine protein phosphatases that are involved in many different cellular processes, including glycogen metabolism, cell cycle regulation, protein synthesis and intracellular transport, ribonucleic acid (RNA) splicing, and signal transduction. Specifically, many important signal transduction molecules, including protein kinase A (PKA), PKA, PKB/Akt, protein kinase C (PKC), CREB, GSK3, Weel, APC, axin, and mitogen-activated protein (MAP) kinases, are substrates of PP1 and PP2A (Cohen, P. T. 2002. J. Cell Sci. 115:241-256; Janssens, V., et al. 2001. Biochem. J. 353:417-439; Millward, T. A., et al. 1999. Trends Biochem. Sci. 24:186-191; Wera, S., et al. 1995. Biochem. J. 311:17-29). Through dephosphorylation of these signal transduction regulators, PP1 and PP2A positively or negatively regulate multiple cellular signaling pathways. Recent discoveries of mutations of PP2A in human lung, colon, breast, and colorectal cancers and melanomas support the notion that PP2A may function as a tumor suppressor gene (Calin, G. A., et al. 2000. Oncogene 19:1191-1195; Deichmann, M., et al. 2001. Melanoma Res. 11:577-585; Ruediger, R., et al. 2001. Oncogene 20:1892-1899; Ruediger, R., et al. 2001. Oncogene 20:10-15; Sontag, E. 2001. Cell. Signal. 13:7-16; Wang, S. S., et al. 1998. Science 282:284-287).

Prostate cancer remains the second leading cause of cancer mortality among American males. The predominant reason for such high and persistent mortality is the lack of curative therapies for androgen-resistant or steroid sensitive metastatic disease. It is critical to elucidate the molecular mechanisms that underlie the ultimate androgen-resistant state of prostate cancer and to develop effective therapies for this condition. There is therefore a need for a model system in which the nonrelevant androgenic-stimulated gene activities would be normalized. Moreover, a better understanding of the molecular basis of metastasis as well as steroid insensitivity would allow rational efforts toward the development of novel effective anti-metastasic therapy to proceed.

### SUMMARY OF THE INVENTION

The present invention provides methods and assays for screening and identifying agents that will be useful in the treatment and prevention of neoplastic disorders. The methods and assays of the present invention are particularly useful for identifying therapeutic agents for steroid sensitive neoplastic disorders.

### BREIF DESCRIPTION OF THE DRAWINGS

FIG. 1 demonstrates the ability of cav-1 to inhibit Tg-mediated apoptosis in LNCaP prostate cancer cells.

FIG. 2 shows the ability of cav-1 to increase activities of PDK1 and Akt but not PI3-K.

FIG. 3 demonstrates that cav-1 interacts with and inhibits PP1 and PP2A.

FIG. 4 shows the scaffolding domain in cav-1 is required for interaction with and inhibition of PP1 and PP2A and is required for cav-1-mediated survival activities against Tg-induced apoptosis.

FIG. 5 demonstrates that cav-1 selectively increases activities of Akt, PDK1, and p42/44 MAP kinase.

FIG. 6 shows that Akt activities are largely responsible for cav-1-mediated cell survival activities.

FIG. 7 shows the ability of purified PP1 and PP2A enzymes to dephosphorylate Akt in vitro in cav-1-expressing, LY294002-treated LNCaP cell lysate.

FIG. 8 demonstrates the ability of cav-1 to increase the half-lives of phosphorylated Akt (P-Akt) and PDK1 (P-PDK1) in LY294002- or Tg-treated LNCaP cells.

FIG. 9 shows the expression of cav-1 leads to increased phosphorylation of multiple Akt substrates.

FIG. 10 shows the expression of cav-1 in cav-1-negative LNCaP cells leads to increased nuclear translocation of phosphorylated AR (P-AR) in vivo.

FIG. 11 depicts a summary of the role of cav-1 in prostate cancer cells.

### DETAILED DESCRIPTION OF SPECIFIC EMBODIMENTS

In addition to prostate cancer, cav-1, has also been identified as an important metastasis-related gene in various other types of malignancies as well, such as colon cancer, breast cancer, bladder cancer, squamous cell cancers including esophageal squamous cell cancer, papillary carcinoma of the thyroid, ovarian cancers, myeloma, pancreatic ductal adenocarcinoma, renal cancer and lung cancer.

cav-1 has been clearly connected with antiapoptotic activities in specific cancer relevant biological contexts, including serum deprivation, androgen withdrawal, and in response to c-myc overexpression. The present disclosure demonstrates, in vivo and in vitro, that cav-1 maintains the phosphorylated state of Akt through scaffolding binding site interactions with and inhibition of two major serine/threonine protein phosphatases, PP1 and PP2A.

The phosphorylation and dephosphorylation of structural and regulatory proteins are major intracellular control mechanisms in eukaryotes. While protein kinases play important roles in intracellular signal transduction, the dynamics of protein phosphorylation is also controlled by protein phosphatases. During normal embryonic development and throughout adult life, intracellular signaling needs to be precisely coordinated and integrated for normal cell functions, including growth.

Oncogenic perturbation can arise not only as a result of overactivation of oncogenic kinases but also as a consequence of obstruction of normal autoinhibitory and regulatory constraints. cav-1 in prostate cancer cells plays a role as a positive regulator in the Akt signaling pathway through the inhibition of negative regulators PP1 and PP2A. Both PP1 and PP2A carry a consensus cav-1 binding motif in their catalytic subunits. cav-1 binding to the catalytic subunits of PP1 and PP2A through the cav-1 scaffolding domain can disrupt the normal catalytic functions of the enzymes and therefore lead to inhibition of PP1 and PP2A. As a consequence, the reduced activities of PP1 and PP2A lead to higher phosphorylation levels of their specific substrates, including PDK1, Akt, and ERK1/2.

Therefore, the present disclosure may also be described, in certain embodiments as methods of identifying agents that exhibit anti-neoplastic activities, or that modulate apoptotic activity in such cells, particularly in steroid responsive cancer cells such as prostate or breast cancer cells, for example. The methods of the present disclosure may include, therefore, the identification of agents that interfere with, or inhibit the interaction of caveolin-1 with the catalytic domains of either PP1, PP2A, or both. Active agents may be identified through assays that measure binding directly, or indirectly through detection of a modulation of the activities of the PP1 and PP2A enzymes. Such methods may include assays conducted in vitro, in a cell culture, for example, or in vivo.

The methods of the disclosure may include, then, a caveolin-1 protein or a fragment thereof comprising the scaffolding domain (amino acids 82-101 of cav-1). Typical binding assays may include the immobilization of a caveolin protein or fragment thereof and contacting the immobilized protein with a PP1 or PP2A enzyme or fragment thereof comprising the catalytic domain. Alternatively, a polypeptide fragment that includes the scaffolding domain may be provided to contact an immobilized PP1 or PP2A enzyme. It is understood that one or both of the binding partners may be labeled with a radioactive, enzymatic or fluorescent label for detection of binding.

In certain embodiments, the disclosed methods may include the use of phosphatase activity assays as described in the examples below. In preferred methods, the phosphatase activity of PP1, PP2A or both may be determined in the presence of caveolin-1 or in the presence of the scaffolding domain of caveolin-1 and further in the presence and in the absence of a candidate substance. A difference in phosphatase activity in the presence and absence of the candidate substance would be an indication that the candidate is a modulator of the caveolin inhibition of phosphatase activity and is thus a viable candidate for a therapeutic agent in the treatment of certain neoplastic diseases or cancers. In yet further embodiments the activity of Akt may be determined as described below. In all such assays, a difference in activity in the presence and absence of a candidate substance or agent is indicative of a modulator of caveolin activity or of caveolin binding to the PP1/PP2A enzymes. The assays are preferably performed in cells such as LnCap cells, and more preferably in cells that express a caveolin-1 gene. The caveolin gene may be introduced into the cell with an adenoviral vector or any other vector known in the art for expression within the cell.

This screening assays described herein include obtaining a candidate substance, which can come from any source. For example, it is proposed that compounds isolated from natural sources such as fungal extracts, plant extracts, bacterial extracts, higher eukaryotic cell extracts, or even extracts from animal sources, or marine, forest or soil samples, may be assayed for the presence of potentially useful pharmaceutical agents. In addition, man made substances which would include, but are not limited to, synthetic peptides, peptide mimetics, or other compounds designed de novo based on the predicted protein structure of the caveolin scaffolding domain, or the PP1 or PPA2 catalytic domains, can also be screened for possible use as pharmaceutical agents. It is also understood that antibodies and other isolated or purified, but naturally occurring compounds could be screened by these processes.

An activated Akt pathway is primarily responsible for cav-1-mediated cell survival activities and indicates that cav-1-mediated MAP kinase pathway activation may contribute to a lesser extent to specific cav-1 stimulated cell survival. Furthermore, while maintaining higher Akt activity, the expression of cav-1 leads to increased phosphorylation of multiple Akt downstream targets, including GSK3a/p, FKHR, and MDM2. Inactivation of GSK3α/β by phosphorylation favors increased β-catenin and c-myc protein levels, since GSK3α/β-mediated phosphorylation leads to ubiquitin-mediated protein degradation in both cases. Phosphorylation of FKHR by Akt disrupts FKHR-mediated transcription of specific proapoptotic genes by promoting export of FKHR from the nucleus to the cytoplasm, resulting in its sequestration by the 14-3-3 protein. Akt-mediated phosphorylation of MDM2 promotes its translocation into the nucleus where it inhibits p53 activities.

Furthermore, in contrast to other cell models expression of cav-1 in cav-1-negative LNCaP cells does not stimulate PI3-K activity but maintains higher activities of PDK1 and Akt two major downstream components of PI3-K, through interaction with and inhibition of two major serine/threonine protein kinases PP1 and PP2A, thereby indicating a cell type-dependent and context dependent function and biological consequences of cav-1 in PI3-K/Akt signaling.

It is interesting that in LNCaP cells Akt is constitutively active due to PTEN mutation. This provides significant advantages for cell survival; however, overexpression of cav-1 also imparts additional protection. Specifically, when these cells are subjected to certain types of experimental stresses, such as thapsigargin (Tg) or LY294002, which can lead to suppression of Akt activities, cav-1-mediated inhibition of PP1 and PP2A provides an unique advantage by further increasing Akt activities and establishing a greater protective barrier. Therefore, overexpression of cav-1 can specifically provide prostate cancer cells, as well as other malignant cells, selection and contextual survival advantages after androgen withdrawal therapy.

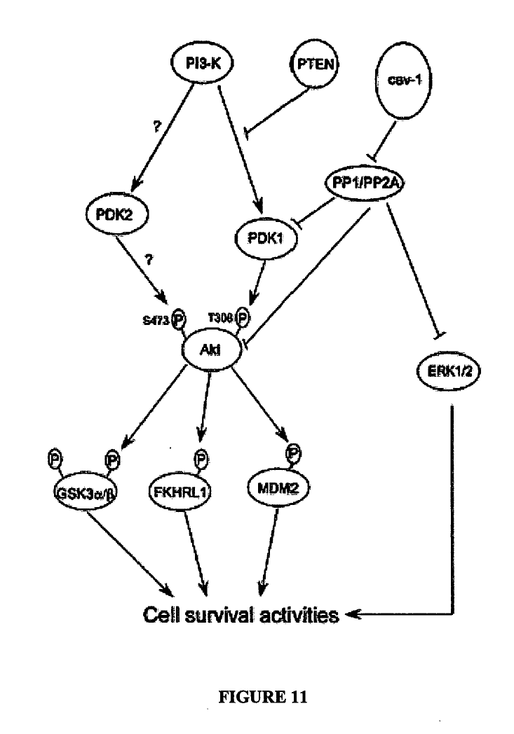

Interestingly, expression of cav-1 leads to increased nuclear translocation of phosphorylated androgen receptor, the crucial event in androgen receptor (AR) action. The increased nuclear translocation of phosphorylated AR by cav-1 provides new insight into the role of cav-1 in hormone-resistant and progressive prostate malignancies. As summarized in FIG. 11, cav-1 overexpression contributes to molecular imbalances that favor malignant progression through its inhibition of PP1 and PP2A. Furthermore, not only is cav-1 expression linked to AR phosphorylation and nuclear translocation but it is also associated with increased testosterone uptake, thereby providing a mechanism to explain the phenomenon of steroid refractory prostate cancer reoccurrence. Therefore, in cells that express or over-express cav-1, agents that disrupt the direct or indirect interaction of cav-1 with AR phosphorylation and/or testosterone uptake will provide beneficial therapeutic effects in the treatment and prevention of androgen refractory prostate cancer cells or for any other type of androgen sensitive cancer type. Furthermore, other steroid interacting molecules (polypeptides or proteins) can also modulate the uptake of steroids or steroid-like substances and a wide variety of cancer types are known in the art to be sensitive or responsive such substances, indicating that agents that disrupt the uptake, binding or internalization of steroids or steroid-like substances as mediated by steroid interacting molecules will likewise provide beneficial therapeutic effects in the treatment and prevention of such cancers.

Steroid interacting molecules as contemplated herein include, but are not limited to, not only cav-1 and cav-like proteins and polypeptides (such as cav-2 and Flotillin), but also members of the steroid receptor superfamily, such as hormone receptors (including glucocorticoid (GR), AR, mineralocorticoid (MR), progesterone (PR), oestrogen (ER), vitamin D (VDR), thyroid hormone (TR), retinoic acid (RAR), retinoid X (RXR)), orphan receptors (including peroxisome proloferator (PPAR), Apo-1 regulatory protein (ARP-1), erb-A related (EAR-1, EAR-2), hepatocyte nuclear factor (HNF-4), steroidogenic Facotr (SF-1), nerve growth factor inducible (NGFI-B), oestrogen receptor related (ERR-1, ERR-2), chicken ovalbumin upstream (COUP)) and Drosophila-related receptor proteins (including ecdysone receptor (EcR), embryonic gonad (EGON), Fushi tarazu factor 1 (FTZ-F1), Knirps (KNI), Knirps related (KNRL), seven-up (SVP), tailless (TLL) and ultraspiracle (USP)). Steroid interacting molecules further comprise any peptides or proteins now-known or in the future discovered to interact directly or indirectly through some type of intermediary molecule, such as fatty acids or glycoproteins, with steroids or steroid-like substances. Steroid or steroid-like substances as used herein refers to substances that have steroid or steroid-like activities or substances that bind to steroid or steroid-like receptors, including but are not limited to androgens such as testosterone, androsterone, progesterone, progestin, and their derivatives. Also included in this group of substances is dehydroepiandrosterone (DHEA), androstenedione (Andro), androstanediol: androsterone, androstenolone, dihydrotestosterone (DHT), testosterone propionate, testosterone enanthate, testosterone cypionate, methyltestosterone, fuoxymesterone, danazol.calusterone, dromostanolone propionate, ethylestranol, methandriol, methandrostenolone, nandrolone decanoate, nandrolone phenpropionate, oxandrolone, oxymetholone, stanozolol, testolactone, estradiol, estriol and estrone, corticosteroids (including glucocorticoids such as cortisol, cortisone and corticosterone, and mineralocorticoids such as aldosterone), as well as cholesterol.

Therefore, in accordance with the findings disclosed herein, the present invention provides methods of identifying agents that modulate the uptake, binding and/or internalization of steroids or steroid-like substances comprising: providing a cell expressing a steroid interacting molecule; contacting the cell with a labeled steroid or steroid-like substance in the presence and absence of at least one agent; and measuring the quantity of steroid or steroid-like substance bound and/or internalized by the cell, wherein a difference in the amount of the steroid or steroid-like substance bound and/or internalized by the cell in the presence of the agent as compared to in its absence is indicative of a modulating agent.

Alternate embodiments of the present invention provide methods for screening and identifying therapeutic agents for the treatment and prevention of neoplastic disorders comprising: expressing a steroid interacting molecule in a cell; contacting the cell with a steroid or steroid-like substance in the presence and absence of at least one agent being screened; and measuring the difference in the uptake of the steroid or steroid-like substance by the cell in the presence and absence of the at least one agent, wherein a change in the amount of steroid or steroid-like substance uptake by the cell in the presence of the at least one agent as opposed to its absence is indicative of a therapeutic agent. While still other embodiments of the present invention provide methods for screening and identifying therapeutic agents for the treatment and prevention of steroid sensitive neoplastic disorders comprising: expressing a steroid interacting molecule in a cell, wherein the cell is a type of steroid sensitive neoplasia; contacting the cell with a labeled steroid or steroid-like substance in the presence and absence of at least one agent; and measuring the difference in steroid or steroid-like substance binding or internalization by the cell in the presence and absence of the at least one agent, wherein a decrease in the amount of steroid or steroid-like substance binding or internalized by the cell in the presence of the at least one agent as compared to its absence is indicative of a therapeutic agent.

In certain embodiments, the methods of the present invention include a wash step to remove excess steroids or steroid-like substances prior to the measuring of the difference in steroid or steroid-like substance uptake, binding and/or internalization by the cell in the presence and absence of the agent(s) being screened.

In certain embodiment of the present invention, testosterone will be utilized as the steroid or steroid-like substance, wherein the testosterone is provided at concentrations ranging from 10⁻⁶ to 10⁻¹⁶ molar, representing concentrations that vary from well above those found in normal physiological environments to below those concentrations that may exist within a host after androgen ablation therapy.

Alternate embodiments of the present invention include methods for screening and identifying therapeutic agents for the treatment and prevention of neoplastic disorders comprising: providing a cell expressing a steroid interacting molecule; contacting the cell with a steroid or steroid-like substance in the presence and absence of at least one agent; and analyzing the phosphorylation of AR, wherein a difference in the amount of AR phosphorylated in the cell in the presence of the agent as compared to in its absence is indicative of a therapeutic agent. Similar embodiments of the present invention comprise: providing a cell which expresses a steroid interacting molecule and contains an AR polypeptide; contacting the cell with a steroid or steroid-like substance in the presence and absence of at least one agent; and analyzing the phosphorylation of the AR polypeptide, wherein a difference in the amount of AR polypeptide phosphorylated in the cell in the presence of the agent as compared to in its absence is indicative of a therapeutic agent. In such embodiments, AR polypeptides may comprise portions of the AR protein or synthetically produced polypeptides of the AR protein or portions thereof which contain the AR phosphorylation sites. Phosphorylation of AR or AR polypeptides may be analyzed using any suitable method known in the art. For example, Western blotting techniques, well known in the art, which utilize AR or AR polypeptide specific antibodies may be used. Such antibodies may, furthermore, be specific for phosphorylated forms of AR or AR polypeptides. Alternative analyzation methods may include feeding radioactive labels, such as ortho-³²P, to the cell prior to its being provided in the methods of the present invention, and then subjecting the cell to lysis and immunoprecipitation through AR or AR polypeptide specific antibodies and subsequently analyzing the immunoprecipitates for the presence of ³²P.

Still other embodiments of the present invention provide in vivo methods of screening and analyzing therapeutic agents for the treatment and prevention of neoplastic disorders comprising: applying a quantity of steroid interacting molecule expressing cells to a defined location in a host animal; applying a quantity of cells lacking expression of the steroid interacting molecule to a different defined location in the host animal; administering an agent to the host animal; administering a steroid or steroid-like substance to the host animal; and analyzing differences in cellular reactions between the cells expressing the steroid interacting molecule and the cells lacking expression of the steroid interacting molecule, wherein differences in the cellular reactions are indicative of a therapeutic agent. Any suitable host animal may be used in these in vivo methods, for example, immunocompromised rodents such as nude mice are suitable hosts. Furthermore, the in vivo methods of the present invention include the use of knock-out mice which have been engineered through methods well known in the art to remove expression of an endogenous steroid interacting molecule. The cellular reactions contemplated in the in vivo methods of the present invention include but are not limited to growth rates, tumor mass, angiogenisis, extravasation, migration, cell killing or death, or apoptosis. The quantity of cells utilized in these in vivo methods will vary according to, among other things, the type of cell utilized but such variations are readily determinable by one of ordinary skill in the art. In preferred embodiments, the quantity of cells provided will be sufficient to generate a primary tumor in the host animal if left untreated by an agent.

Agents identified by and through the methods of the present invention will be prime candidate therapeutics for the treatment of cancer, especially steroid sensitive cancers such as prostate cancer, and include naturally occurring or synthetic proteins, peptides, non-peptide small molecules, and any other source of therapeutic candidate agents, such as, for example, specific steroid antagonists, membrane components that can act as intermediates in the interaction of steroid interacting molecules and steroid or steroid-like substances, and antagonists that effect the direct or indirect interactions of cav-1 with steroid or steroid-like substances. In certain embodiments, the agent may even be cav-1 proteins for portions thereof. Agents identified as affecting steroid or steroid-like substance uptake, binding and/or internalized may be subsequently tested for biological activity and used as therapeutics or as models for rational drug design.

The cell utilized in the methods of the present invention may express an endogenous steroid interacting molecule, may be induced to upregulate expression of an endogenous endogenous steroid interacting molecule or express an exogenous steroid interacting molecule. Furthermore, the cell of the present invention may have an endogenous steroid interacting molecule eliminated from its genome by knock-technology and methods (or other similar molecular techniques) or blocked from expression (by any number of techniques well known in the art, such as anti-sense RNA, siRNA, or other transcriptional or translational controls). In some embodiments the cell will then be induced to express an exogenous steroid interacting molecule. In embodiments in which the cell is not induce to express an exogenous steroid interacting molecule, the present invention provides methods of screening and identifying agents that modulate the uptake, binding and/or internalization of steroids or steroid-like substances comprising: providing an altered cell which does not express a given steroid interacting molecule; providing and unaltered cell which does express the given steroid interacting molecule; contacting both the altered cell and the unaltered cell with a steroid or steroid-like substance in the presence of at least one agent; and measuring the cellular responses of both the altered cell and unaltered cell, wherein a difference in the cellular responses is indicative of a modulating agent. The cellular reactions contemplated these methods of the present invention include but are not limited to uptake, binding and/or internalization of steroid or steroid-like substances, growth rates, tumor mass, angiogenisis, extravasation, migration, cell killing or death, or apoptosis.

Any suitable expression system may be employed for the cell of the present invention. As such, the cell provided may be stably or transiently transformed or transfected with any suitable expression vectors containing a nucleotide sequence encoding a steroid interacting molecule or portion thereof, wherein one of skill in the art will readily recognize that certain non-essential modifications may be made in the cav-1 encoding sequence without effecting the utility of the methods of the present invention.

Established methods for introducing DNA into mammalian cells have been described (Kaufman, R. J., Large Scale Mammalian Cell Culture, 1990, pp. 15-69). Additional protocols using commercially available reagents, such as Lipofectamine lipid reagent (Gibco/BRL) or Lipofectamine-Plus lipid reagent, can be used to transfect cells (Felgner et al., Proc. Natl. Acad. Sci. USA 84:7413-7417, 1987). In addition, electroporation can be used to transfect mammalian cells using conventional procedures, such as those in Sambrook et al. (Molecular Cloning: A Laboratory Manual, 2 ed. Vol. 1-3, Cold Spring Harbor Laboratory Press, 1989). Selection of stable transformants can be performed using methods known in the art, such as, for example, resistance to cytotoxic drugs. Examples of selectable markers that can be incorporated into an expression vector include cDNAs conferring resistance to antibiotics, such as G418 and hygromycin B. Cells harboring the vector can be selected on the basis of resistance to these compounds.

Transcriptional and translational control sequences for mammalian host cell expression vectors can be excised from viral genomes. Commonly used promoter sequences and enhancer sequences are derived from polyoma virus, adenovirus 2, simian virus 40 (SV40), and human cytomegalovirus. DNA sequences derived from the SV40 viral genome, for example, SV40 origin, early and late promoter, enhancer, splice, and polyadenylation sites can be used to provide other genetic elements for expression of a structural gene sequence in a mammalian host cell. Viral early and late promoters are particularly useful because both are easily obtained from a viral genome as a fragment, which can also contain a viral origin of replication (Fiers et al., Nature 273:113, 1978; Kaufman, Meth. in Enzymology, 1990). Furthermore, the vector used to express the steroid interacting molecule may be placed under any number of a form of inducible promotor systems, which are well known to those of skill in the art.

The cell used in the methods of the present invention may be any type of mammalian or yeast cell. In certain embodiments, the cell will express either endogenous or exogenous AR. In other embodiments the cell will be from a cancer cell line, preferably a cancer cell line from a steroid sensitive or steroid responsive cancer type. In some embodiments, the cell will be selected from a group consisting of: prostatic cancer, breast cancer, colon cancer, bladder cancer, squamous cell cancer (including esophageal squamous cell cancer), papillary carcinoma of the thyroid, ovarian cancers, myeloma, pancreatic ductal adenocarcinoma, renal cancer and lung cancer. In some of these embodiments, the cell used will be from the prostate cancer cell lines LNCaP or LAPC-9 or the breast cancer cell lines T47D or MCF7. While in still other embodiments, the cell utilized will be a cell which is responsive to steroids or steroid-like substances, especially androgens.

The steroid or steroid-like substance used in the methods of the present invention may be labeled in any fashion which will allow for its identification for the purposes of measuring its uptake, binding and/or internalization. Several methods, which are well known in the art, provide mechanisms by which such substances may be radiolabeled. For example, testosterone may be labeled with ¹⁴C or ¹²⁵I. Other methods known in the art, label testosterone through a photoaffinity mechanism with ³H. Furthermore, the methods of the present invention may utilize steroid or steroid-like substances labeled with colorimetric or fluorescent moieties or attachments, whether directly or indirectly attached. The methods of measuring such labels as contemplated by the present invention are well known to those of skill in the art.

The following nonlimiting examples serve to further illustrate the present invention.

### EXAMPLES

### Materials and Methods for Examples

Cell Lines, Antibodies, and Reagents:

The human prostate cancer cell line LNCaP was obtained from the American Type Culture Collection, grown in RPMI 1640 medium with 10% fetal calf serum (FTS), and used at passage 30 to 50. Monoclonal antibodies against PI3-K (clone 4), PKBα/Akt (clone 55), caspase 3 (clone 19), caspase 7 (clone B94-1), PP1 catalytic subunit (PP1-C) (clone 24), PP2A catalytic subunit (PP2A-Ca) (clone 46), and androgen receptor (AR) (clone G122-77) were purchased from BD Biosciences. Monoclonal anti-α-actin (clone AC-15) and protease inhibitor cocktail were from Sigma. Rabbit polyclonal antibodies against cav-1 (N-20), PP1-C (FL-18), PKB kinase/PDK1 (H-328), and epidermal growth factor receptor (EGFR) (1005), mouse monoclonal antibodies GSK3β (0011-A) and MDM2 (SMP14), and goat polyclonal antibodies FKHRL1 (N-16), PP2A-Aα subunit (C-20) were obtained from Santa Cruz Biotechnology. Rabbit polyclonal antibodies against IKKα, IKKβ, phospho-Akt (Ser473), phospho-Akt (Thr308), phospho-IKKα (Ser180)/IKKβ (Ser181), phospho-MDM2 (Ser166), phospho-FKHR (Thr24)/FKHRL1 (Thr32), phospho-tyrosine (P-Tyr-100), monoclonal antibody against phospho-CREB (Ser133), and Akt kinase assay kit were from Cell Signaling. Purified rabbit PP1 catalytic subunit was from New England BioLabs. Anti-PI3-K p85 rabbit antiserum, rabbit polyclonal anti-PP2A-C subunit, mouse monoclonal anti-PP2A-B (PR55) subunit, purified human PP2A core enzyme, PDK1 immunoprecipitation kinase assay kit, Ser/Thr phosphatase assay kit 1, and BAD (Ser112/136) phosphorylation detection kit were from Upstate Biotechnology. Antibodies recognizing cleaved caspases 3 and 7 were purchased from Oncogene. Horseradish peroxidase (HRP)-conjugated goat anti-mouse antibodies and HRP-conjugated goat anti-rabbit antibodies were from ICN/Cappel. Tg, LY294002, U0126. and PD98059 were from Calbiochem. CellTiter 96 aqueous one solution reagent was from Promega. [γ-³²P]ATP was purchased from ICN. Flexible plates for thin-layer chromatography (TLC) was obtained from Whatman. His-V5-tagged human cav-1 plasmid vector was constructed, and its protein was produced and purified as described previously (Tahir, S. A., et al. 2003. Clin. Cancer Res. 9:3653-3659).

Transfection and Viral Infection:

The plasmid vector expressing wild-type cav-1 (pcav-1) was constructed by inserting the human cav-1 cDNA into pcDNA3.1. Mutant cav-1 with the scaffolding domain deleted (cav-1Δ82-101) was generated by PCR mutagenesis using pcav-1 as a template. In the first step, intermediate PCR product A was produced using T7 promoter primer 5′CTGAGTGATATCCC3′ and primer A carrying an adjacent DNA sequence from both sides of the region deleted (5′CAGACAGCAAAAAACTGTGT3′), and intermediate PCR product B was produced using primer B which also carries a DNA sequence adjacent to both sides of the region deleted (5′TGTGTCAAAAAACGACAGAC3′) and pcDNA3.1/BGH reverse primer (5′TAGAAGGCACAGTCGAGG3′). These two intermediate PCR products which carry 20-mer overlapped DNA sequence were annealed and amplified using T7 promoter primer and pcDNA3.1/BGH reverse primer. The resulting PCR product was digested with EcoRI and inserted into pcDNA3.1 (+) to generate pcav-1Δ82-101. The full sequence was confirmed by sequencing using an automatic sequencer (ABI Prizm 310). Dominant-negative Akt1 (K179M) cDNA expression kit was from Upstate Biotechnology and dominant-negative Akt1 (T308A, S473A) and PTEN expression vectors were described previously (Wen, Y., et al. 2000. Cancer Res. 60:6841-6845). Recombinant adenoviral vectors Adcav-1 and AdRSV were generated as described previously (Nasu, Y., et al. 1998. Nat. Med. 4:1062-1064, Timme, T. L., et al. 2000. Oncogene 19:3256-3265). Subconfluent cells were trypsinized, collected by centrifugation, and resuspended in regular medium. A single-cell suspension was then seeded at 5×10⁵ cells/well (six-well plates) or 2×10⁶ cells/10-cm-diameter plate. Cells were infected or transfected the next day. Typically, cells were infected with Adcav-1 or AdRSV in serum-free medium (SFM) at a multiplicity of infection (MOI) of 10. The infection medium was removed and replaced with complete medium 3 h after the infection. For transfection, 2 μg of DNA was used for each transfection in the six-well plates using Promega Tfx-50 reagent at the Tfx-50/DNA ratio of 2:1 in 1 ml of SFM, and 12 μg of DNA was used to transfect cells in a 10-cm diameter plate with 5 ml of SFM. One hour after the transfection, two milliliters of RPMI 1640 medium with 15% FCS was added to each well (six-well plate) or 5 ml of RPMI 1640 medium with 20% FCS was added to each 10-cm diameter plate. For experiments with both infection and transfection, cells were transfected 16 h (overnight) after the viral infection.

Viability Assay and Apoptosis Analysis:

Cells were infected or transfected in six-well plates. Forty-eight hours after infection or transfection, cells were treated with 1 μM Tg for the time indicated. At the end of the treatments MTS assays were performed using CellTiter 96 aqueous one solution reagent from Promega by a modified version of the procedure. Briefly 300 μl of MTS solution was added to each well and incubated for 1 h at 37° C. One milliliter of reaction or medium mix was transferred from each well to an Eppendorf tube and then centrifuged in a microcentrifuge for 5 min at 4,000 rpm (˜3,000×g). One hundred microliters of supernatant was transferred to each well of a 96-well plate, and the relative viability was determined by the measurement of absorbance at 490 nm in a 96-well plate reader. In viral infection experiments, a moderate level of cav-1 was generated at an MOI of 10 and the difference in cell proliferation between Adcav-1- and AdRSV-infected cells was minimal; therefore, the difference derived from MTS staining mainly represents cell viability. Apoptotic morphology was analyzed with phase-contrast fluorescence microscopy after incubation with 0.2 μg of 4′6′-dismidino-2-phenylindole (DAPI) per ml. The activities of caspases in the cell lysates were analyzed by Western blotting using specific antibodies recognizing procaspases or cleaved forms of caspases, followed by quantitative intensity analysis using software (GelExpert; Nucleo Vision) and normalized to the intensity of β-actin in the same sample.

IP and Immunoblotting:

Unless specifically indicated, immunoprecipitation (IP) was performed as follows. Cells were washed with ice-cold phosphate-buffered saline (PBS), lysed on the plates by incubation for 10 min on ice with IP lysis buffer, which consists of 20 mM Tris-HCl (pH 7.5), 150 mM NaCl, 1 mM EDTA, 1 mM EGTA, 1% Triton X-100, 1 mM β-glycerophosphate, 60 mM octylglucoside and protease inhibitor cocktail. Cells were then scraped off the dish, transferred to microcentrifuge tubes, placed on ice for 20 min, and centrifuged for 10 min at high speed in a microcentrifuge at 4° C. The supernatant (cell lysate) was transferred to a fresh tube and frozen at −80° C. until use. Typically, 500 μg of cell lysate proteins were diluted to 500 μl with IP lysis buffer and precleared by incubation with 3 μg of the corresponding immunoglobulin G (IgG) from healthy cells for 30 min at 4° C. on a rocker, followed by the addition of 50 μl of IP lysis buffer-prewashed protein A or G plus agarose and incubation for another 1 h. The cleared cell lysate was incubated with 3 μg of specific antibody or the corresponding IgG overnight at 4° C. on a rocker. The immunocomplex was captured by the addition of 50 μl of IP lysis buffer-prewashed protein A or G plus agarose and incubation for another 2 h. The agarose beads were washed three times with IP lysis buffer and resuspended in 40 μl of 2× sample buffer. Ten microliters of each sample containing immunocomplex was separated by electrophoresis on a sodium dodecyl sulfate (SDS)-12% polyacrylamide gel, transferred to a nitrocellulose membrane, and blotted with specific antibodies. For regular Western blot analysis, protein concentration in cell lysates was determined by the Bio-Rad protein assay, and 30 μg of protein from each sample was loaded on the gel. β-Actin served as a loading control. For quantitative analyses, the intensity of protein bands was determined by densitometry (GelExpert; NucleoVision) and normalized to the intensity of β-actin in the same sample.

Phosphatase Activity Assay:

Cells were lysed on the plates with phosphatase lysis buffer containing 20 mM HEPES (pH 7.4), 10% glycerol, 0.1% Nonidet P-40 (NP-40), 1 mM EGTA, 30 mM β-mercaptoethanol, 1 mM phenylmethylsulfonyl fluoride (PMSF), 2 μg of leupeptin per ml, and aprotinin. PP1-C or PP2A-C IP complexes were prepared as described above, except that different lysis buffers and a shorter incubation (1 h) of the lysate and antibody were used. The agarose beads were washed twice with phosphatase lysis buffer and once with phosphatase assay buffer (50 mM Tri-HCl [pH 7.0], 100 mM CaCl₂). Activities of PP1 and PP2A were determined by using a malachite green phosphatase assay protocol with a phosphopeptide (K-R-pT-I-R-R) as the substrate (Upstate Biotechnology), followed by the measurement of absorbance at 620 nm.

Akt Activity Assay:

The cells were washed with ice-cold PBS and lysed on the plates with Akt kinase lysis buffer (Cell Signaling). The Akt activity assay was performed according to the manufacturer's suggested protocol (Cell Signaling). Ten microliters of each resulting reaction product in SDS sample buffer was loaded on a SDS 12% polyacrylamide gel. Phosphorylation of GSK3 was determined by Western blotting with anti-phospho-GSK3α/β (Ser9/21) antibody.

PI3-K Activity Assay:

Cell lysates were prepared as described above for the Akt activity assay. PI3-K activity assays were performed as described below. Briefly, 500 μg of cell lysate proteins were immunoprecipitated with monoclonal antibody against phosphotyrosine for 3 h at 4° C. with gentle rocking, and the beads were washed twice with lysis buffer and twice with PI3-K assay buffer (20 mM Tris [pH 7.5], 100 mM NaCl, 0.5 mM EGTA, 20 mM MgCl₂). PI was dissolved in chloroform (5 mg/ml) and diluted to 0.5 mg/ml with PI3-K assay buffer, followed by sonication. The washed beads were resuspended in 20 μL of PI3-K assay buffer, 20 μl of sonicated PI was added, and the reaction was initiated by adding 10 μl of ATP solution (50 μM ATP and 10 μCi of [γ-³²P] ATP in PI3-K assay buffer). After 15-min incubation at room temperature in a shaking incubator, the reaction was terminated by adding 150 μl of chloroform-methanol-concentrated HCl (50:100:1), and the lipid was extracted after addition of 100 μl of chloroform. The chloroform phase was washed with 200 μl of methanol-1 N HCl (1:1). Five microliters of washed chloroform phase from each sample was loaded on a TLC plate and analyzed by ascending chromatography in chloroform-methanol-29.5% ammonium hydroxide-water (90:90:8:19), followed by autoradiography.

PDK1 Activity Assay:

Cell lysates were prepared as described above. PDK1 activity assay was performed according to the manufacturer's protocol (Upstate Biotechnology). Radioactivity that remained on the P81 paper was read with TopCount (Packard).

IP Using Purified cav-1, PP1, and PP2A Proteins:

Five hundred nanograms of purified cav-1 protein was incubated with 500 ng of purified PP1 (catalytic subunit) or PP2A (core enzyme) in 100 μl of TNES lysis buffer (50 mM Tris [pH 7.5], 100 mM NaCl, 2 mM EDTA, 1% NP-40, protease inhibitor cocktail) for 1 h at 4° C. on a rocker. One microgram of healthy rabbit IgG or rabbit polyclonal antibodies specific for cav-1 or the PP1-C or PP2A-Cα subunit was added, and incubation was continued overnight at 4° C. on a rocker. The immunocomplexes were captured by the addition of 20 μl of prewashed protein A or G plus agarose and incubation for another 2 h.

Effects of cav-1 on PP1 and PP2A Activities:

PP1 (catalytic subunit) (0.02 U) or PP2A (core enzyme) (0.01 U) was incubated with or without 100 ng of purified cav-1 protein for 1 h at 4° C. Phosphatase assay buffer and a phosphopeptide (K-R-pT-1-R-R) were added to a final volume of 50 μl. Reactions were initialed by the addition of 200 μM phosphopeptide, and reaction mixtures were incubated at room temperature for 10 min. Reactions were terminated by the addition of 100 μl of malachite green solution. Activities of PP1 and PP2A were determined by the measurement of absorbance at 620 nm.

Subcellular Distribution of cav-1, PP1, and PP2A in cav-1-Expressing LNCaP Cells:

Adcav-1-infected LNCaP cells were washed once with ice-cold PBS, scraped from plates in ice-cold homogenization buffer (10 mm HEPES [pH 7.9], 10 mM KCl, 1.5 mM MgCl₂, 0.2 mM EDTA, 0.2 mM EGTA, 1 mM β-glycerophosphate, 0.5 mM dithiothreitol [DTT], 1 mM Na₃VO₄, protease inhibitor cocktail), and incubated on ice for 15 min. Cells were disrupted mechanically by being passed 20 times through a 26⅜ gauge needle. Nuclei, mitochondria, and unbroken cells were removed after centrifugation at 100,000×g for 10 min at 4° C. The microsomal fraction was obtained after centrifugation of 10,000×g supernatant at 100,000×g for 1 h at 4° C. The 100,000×g supernatant was designated the cytosolic fraction. The distributions of cav-1, PP1, and PP2A in the microsomal fraction and cytosol were examined by Western blotting using specific antibodies against cav-1, PP1-C, or PP2A-Cα. EGFR was used as a marker for the plasma membrane.

Effects of PP1 or PP2A on cav-1-Mediated Regulation of Akt Phosphorylation:

Fifty micrograms of cav-1-infected LNCaP cell lysate proteins were incubated for 30 min at 30° C. with purified PP1 or PP2A at the concentrations indicated in a protein phosphatase assay buffer (Upstate Biotechnology) or in a PP1-specific assay buffer (with 1 mM MnCl₂; New England BioLabs) in a final volume of 20 μl. The reactions were terminated by adding 4 μl of 6×SDS sample buffer and heating for 5 min in a boiling water bath. The effects of PP1 or PP2A on cav-1-mediated regulation of Akt phosphorylation were analyzed by Western blotting using phospho-specific antibodies for Akt at both sites S473 and T308. Total Akt was also evaluated as a loading control.

Time Course Analyses for Phospho-Akt and Phospho-PDK1:

Forty-eight hours after infection, Adcav-1-infected cells or AdRSV-infected cells were treated with 1 μM Tg or 20 μM LY294002 for the times indicated. The cells were washed with ice-cold PBS and lysed on the plates with Akt kinase lysis buffer. Western blotting was performed as described above. For quantitative analyses, the intensity of interested protein bands was determined by using GelExpert software (NucleoVision) and normalized to the intensity of β-actin in the same sample. Half-life was obtained from equations generated by fitting a line to the data points.

In Vivo Phosphorylation of AR:

LNCaP cells were infected with Adcav-1 or AdRSV at an MOI of 10. Forty-eight hours after infection, cells were treated for 30 min with 20 μM PI3-K inhibitor LY294002 in complete RPMI 1640 medium. Cells were washed once with phosphate-free serum-free RPMI 1640 medium and incubated in the same medium for 30 min. The cells were then incubated for 6 h in phosphate-free serum-free RPMI 1640 medium containing 5 pM or nM DHT and 200 μCi of [³²P per ml.] Radioactive medium was removed, and cells were washed three times with ice-cold PBS, lysed on the plate with a modified version of the ice-cold homogenization buffer (10 mM HEPES [pH 7.9] 10 mM KCl, 1.5 mM MgCl2, 0.2 mM EDTA, 0.2 mM EGTA 0.5 mM DTT, 1 mM β-glycerophosphate 1 mM Na₃VO₄, 1 mM PMSF 1 μg of leupeptin per ml), and incubated on ice for 15 min. The cells were then mechanically disrupted b3 being passed 20 times through a 26⅜-gauge needle, and the lysate was centrifuged for 5 min at at 800×g. The resulting soluble fraction was saved as the cytosolic fraction, and the resulting nuclear pellet was resuspended in a modified version of the ice-cold nuclear extract buffer (20 mM HEPES [pH 7.9], 0.4 M NaCl, 1.5 mM MgCl₂, 0.2 mM EDTA, 0.2 mM EGTA, 0.5 mM DTT, 25% glycerol, 1 mM ⊕-glycerophosphate 1 mM Na₃VO₄ 1 mM PMSF, 1 μg of leupeptin per ml), incubated on ice for 30 min, and centrifuged for 10 min at high speed to remove insoluble materials. The resulting nuclear extract and the cytosolic fraction were precleared by incubation with healthy mouse IgG and protein A or G plus agarose for 2 h. The cleared nuclear extract and cytosolic fraction were incubated overnight at 4° C. with a mouse monoclonal antibody specific for AR, followed by an incubation with protein A or G plus agarose for 2 h. The immunocomplexes were washed three times with IP lysis buffer and then resuspended in 30 μl of SDS sample buffer. After the separation on an SDS-10% polyacrylamide gel, total AR was analyzed by Western blotting with a polyclonal antibody specific for AR, and the phosphorylated AR was analyzed by autoradiography.

### Example 1

### cav-1 Protects Cells from Tg-Mediated Cell Death

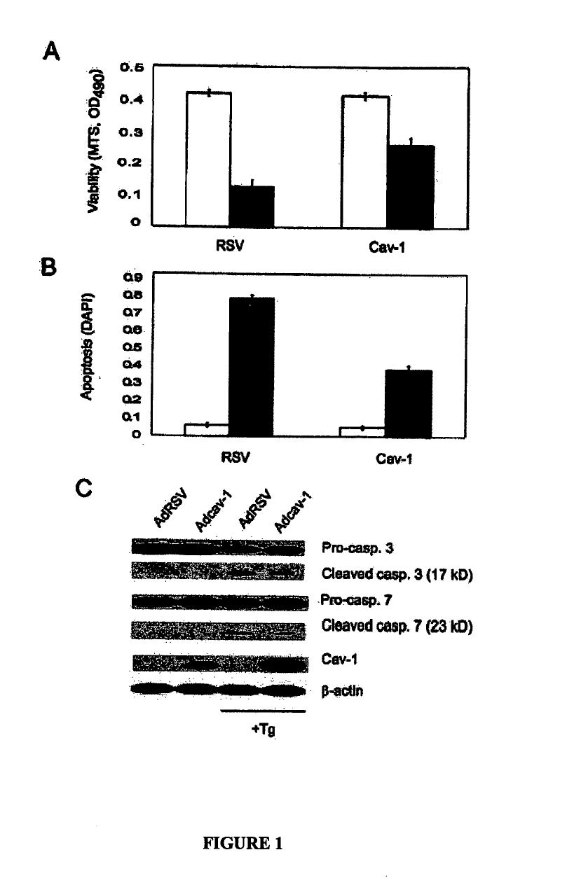

LP-LNCaP cells (cav-1 negative) were infected with a cav-1-expressing adenoviral vector (Adcav-1) or a control adenoviral vector carrying only the respiratory syncytial virus (RSV) promoter (AdRSV) at a MOI of 10. Forty-eight hours after infection, cells were treated with 1 μM Tg or not treated with Tg for 24, 32, or 48 h. Cell viability of Adcav-1 or AdRSV-infected cells with or without Tg treatment was evaluated by measuring the absorbance or optical density at a wavelength of 490 nm (OD490) in an MTS staining assay (FIG. 1A). Forty-eight hours after Tg treatment, cav-1-infected cells maintained ˜62% viability relative to Tg-untreated cells, whereas control RSV-infected cells were ˜30% viable. The results show that the expression of cav-1 increased cell viability approximately twofold compared with control AdRSV-infected cells.

To verify cell protection by cav-1 against Tg-induced apoptosis, apoptosis was evaluated in cav-1-expressing cells and AdRSV-infected control cells by quantitative analyses of DAPI-stained apoptotic and no-apoptosis nuclei. In addition, the activities of two major executor caspases, caspases 3 and 7, which have been widely used as markers for apoptosis, were also analyzed by Western blotting using specific antibodies recognizing procaspases (Pro-casp.) (inactive) or cleaved caspases (casp.) (active). Tg-induced apoptosis initiated 24 h after the treatment became obvious at 32 h and more extensive at 48 h. Apoptotic cells or nuclei were significantly reduced in cav-1-expressing cells. FIG. 1B documents that 48 h after the treatment with Tg, the expression of cav-1 suppressed Tg-induced apoptosis from 76 to 37% (expressed as the ratio of apoptotic nuclei to total nuclei). In untreated control cells, caspases 3 and 7 were present in procaspase forms. Forty-eight hours after Tg treatment, procaspases were reduced, and cleaved active caspases 3 and 7 were increased in the cells. As determined by the levels of cleaved active caspases, quantitative analysis demonstrated decreases of ˜40 to 50% in caspase 3 and caspase 7 activities in cav-1-expressing cells relative to RSV-infected cells. These data show that cav-1 is capable of protecting cells from Tg-induced apoptosis.

### Example 2

### cav-1 Increases PDK1 and Akt Kinase Activity

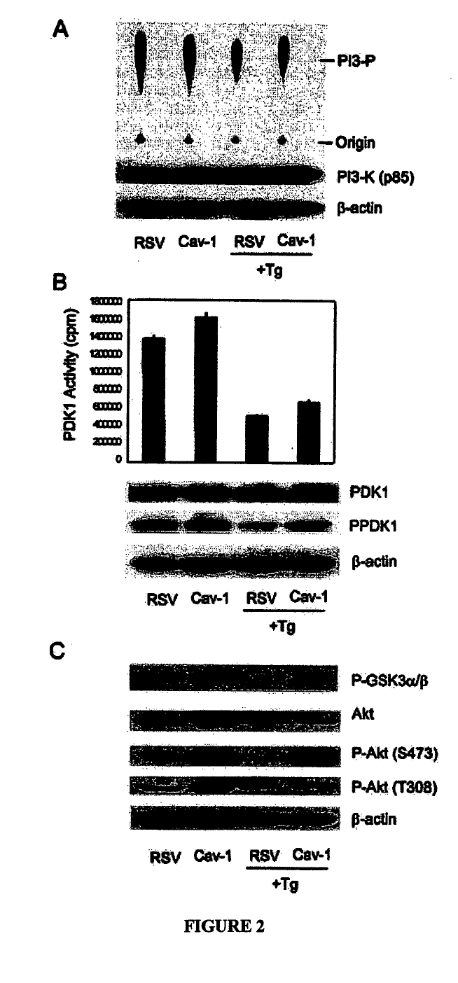

Protein expression levels and kinase activities of PI3-K, PDK1, and Akt were determined in cav-1-expressing or vector control LNCaP cells before and after treatment with 1 μM Tg for 48 h (+Tg): (A) PI3-K kinase assay by TLC (top gel); (B) PDK1 IP kinase assay; (C) Akt IP kinase assay using recombinant GSK3 as a substrate. Total protein and phosphorylated protein (indicated by P-prefix before protein name) of each kinase were analyzed and presented in each blot. β-Actin served as a loading control. There was no difference in protein expression levels for PI3-K in cav-1-expressing cells to control vector-infected cells (FIG. 2A). Although PI3-K activity was reduced by Tg treatment, no difference was found between cav-1-expressing and control cells (FIG. 2A). Tg treatment also led to reduced PDK1 activity; however, compared with control RSV-infected cells, PDK1 activity was approximately 17% higher in cav-1-expressing cells and approximately 30% higher in Tg-treated cav-1-expressing cells (FIG. 2B). In the same lysates, there was no difference in PDK1 protein expression (FIG. 2B). After Tg treatment, total Akt protein level showed significant reduction, presumably due to proapoptotic activities of Tg that can involve reduction of protein synthesis and protein degradation; however, significantly higher Akt kinase activity (determined by phosphorylation of its substrate GSK3) was observed in cav-1-expressing cells. Akt kinase activity was approximately twofold higher in cav-1-expressing cells and was about eightfold higher in Tg-treated cav-1-expressing cells compared with their corresponding control cells (FIG. 2C). Thus, the results indicate that cav-1-mediated survival activities were not a result of higher PI3-K activities but were derived from increased PDK1 and Akt kinase activities.

### Example 3

### cav-1 Interacts with and Inhibits PP1 and PP2A In Vivo and In Vitro

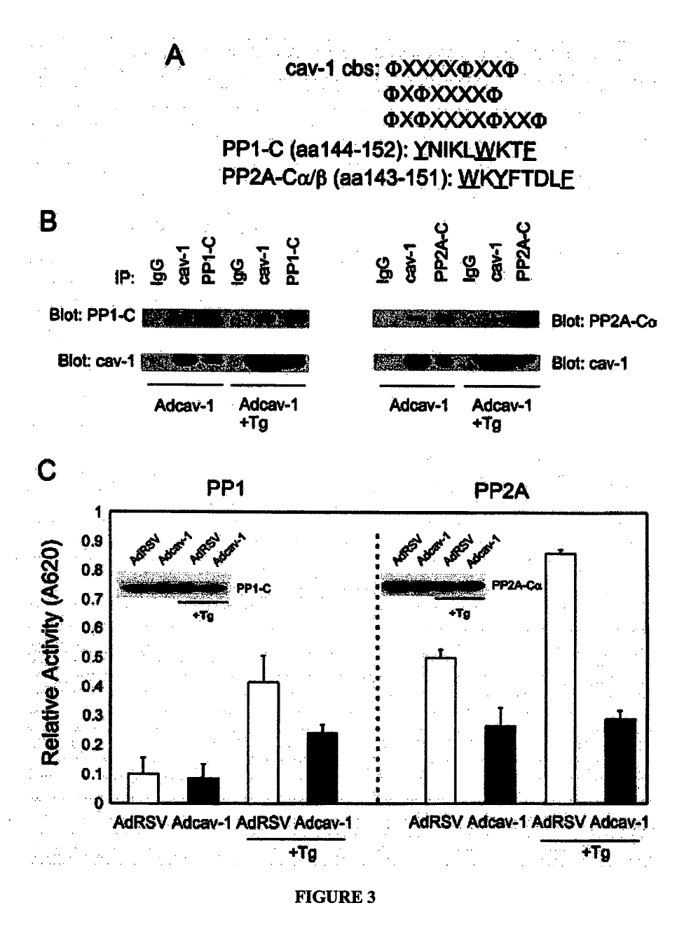

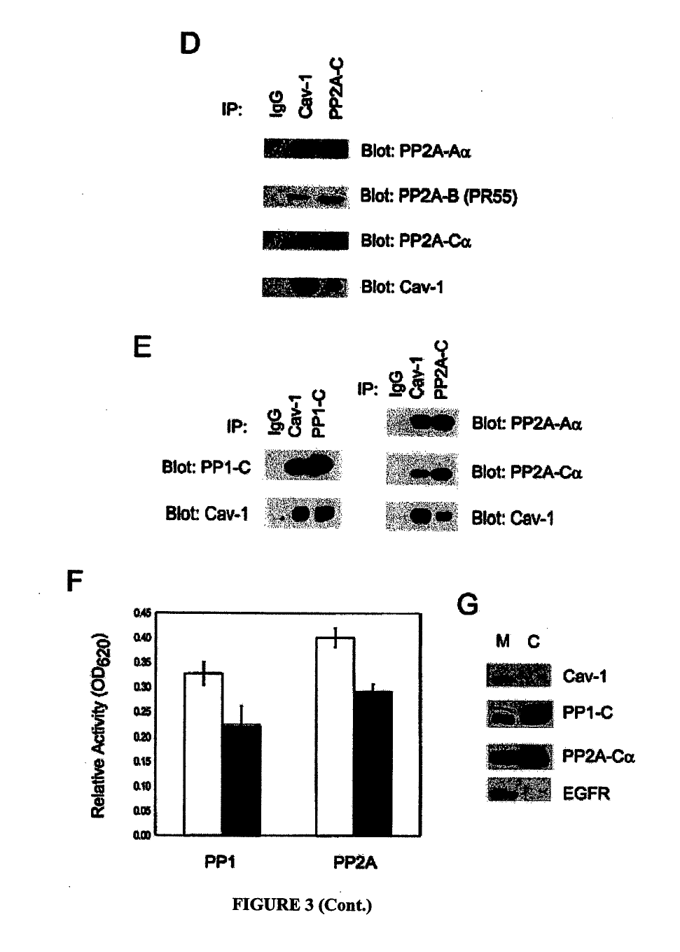

Both catalytic subunits of PP1 and PP2A possess a previously described consensus caveolin binding site (FIG. 3A: aromatic residues (Φ) and any amino acid (X) in the cav-1 consensus binding sites are indicated). IP experiments demonstrated that cav-1 is present in PP1-C or PP2A-Cα IP complexes and PP1-C or PP2A-Cα is also found in the cav-1 IP complex (FIG. 3B), indicating that cav-1 can bind to PP1 or PP2A. Importantly, serine/threonine protein phosphatase activity assays using PP1-C or PP2A-Cα IP complexes and a synthetic phosphopeptide substrate indicated that interaction with cav-1 leads to the inhibition of PP1 and PP2A activities (FIG. 3C: relative activity was determined by measuring the absorbance at 620 nm (A620)). Reduced overall activities of PP1 and PP2A favor the maintenance of Akt phosphorylation and increased Akt activity. Inserted blots in FIG. 3C show expression of PP1-C and PP2A-Cα in the same cell lysates.

### Example 4

### Both PP1 and PP2A Consist of Multiple Subunits and May Exist in Different Forms

PP2A can exist as free catalytic subunit (C subunit), core enzyme (A and C units), or holoenzyme (A, B, and C subunits). Although the amino acid sequences of PP1 and PP2A showed that both their catalytic subunits possess a previously described consensus caveolin-1 binding site, indicating that cav-1 may interact with C subunits of PP1 and PP2A, and coimmunoprecipitation of cav-1 and PP1-C or PP2A-Ca has been demonstrated above, the form(s) of PP1 and PP2A with which cav-1 interacts had not been fully demonstrated in an experimental model. To address this question for PP2A, the A and B subunits of PP2A were probed for using antibodies specific for the isoform of the A subunit of PP2A (PP2A-Aα) and for the B subunit (PR55) of PP2A [PP2A-B (PR55)] in cav-1 or PP2A-Cα IP complexes. The results show that PP2A-Aα and PP2A-B (PR55) are present in cav-1 or PP2A-Cα IP complexes (FIG. 3D), indicating that cav-1 may associate with PP2A holoenzyme. In vitro IP experiments using purified PP2A core enzyme (FIG. 3E) demonstrated that cav-1 can interact with PP2A core enzyme and that PP2A-Aα is present in cav-1 or PP2A-C IP complexes. In vitro IP experiments using free PP1-C demonstrated that cav-1 can interact with PP1-C outside the context of holoenzyme. Overall, this data indicates that cav-1 binds directly to catalytic subunit (C subunit) of PP2A within the context of holoenzyme; however, the possibility remains that cav-1 interacts with free catalytic subunit.

To determine whether the effects of cav-1 on PP1 and PP2A are direct and whether cav-1 interacts with and inhibits PP1 and PP2A in vitro, in vitro IP experiments were performed using purified His-V5-tagged human cav-1, PP1 (C subunit), and PP2A (core enzyme). The data presented in FIG. 3E shows that cav-1 binds effectively to PP1-C or PP2A core enzyme in vitro, indicating that the effects of cav-1 on PP1 and PP2A are direct. The results of in vitro phosphatase assays indicate that the addition of cav-1 reduced PP1 and PP2A activities by approximately 30% under these basic conditions (FIG. 3F). Briefly, cells were treated with purified cav-1 (¢) or not treated with purified cav-1 (£) and relative activity was determined by measuring the optical density at 620 nm (OD620).

To analyze the compartmentalization of cav-1-PP1 and cav-1-PP2A interactions, the subcellular distribution of cav-1, PP1, and PP2A were determined. These results reveal that cav-1 is present exclusively in the membrane, while PP1 and PP2A are present in both the cytosol and membrane (FIG. 3G: microsomal fraction (M) (100,000×g pellet) and cytosol fraction (C) (100,000×g supernatant); EGFR was used as a marker for the plasma membrane). These results indicate that cav-1-bound phosphatases are membrane bound, and the interactions of cav-1 with PP1 or PP2A potentially occurs in the membrane.

### Example 5

### cav-1 Interacts with and Inhibits PP1 and PP2A Through Scaffolding Domain Binding Site Interactions with Catalytic Subunits of PP1 and PP2A

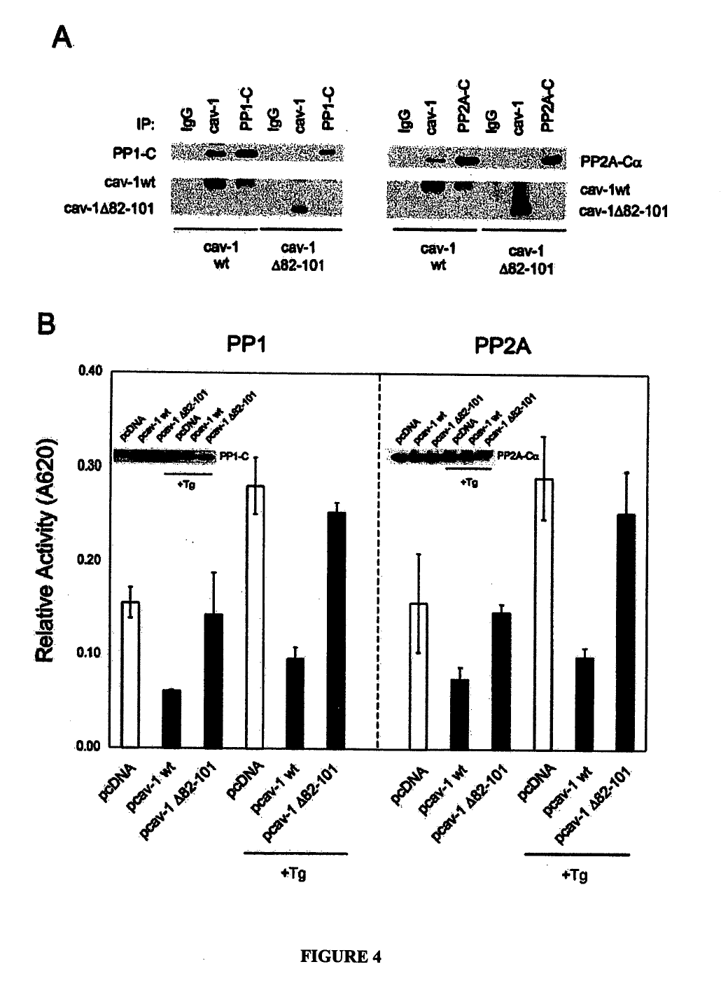

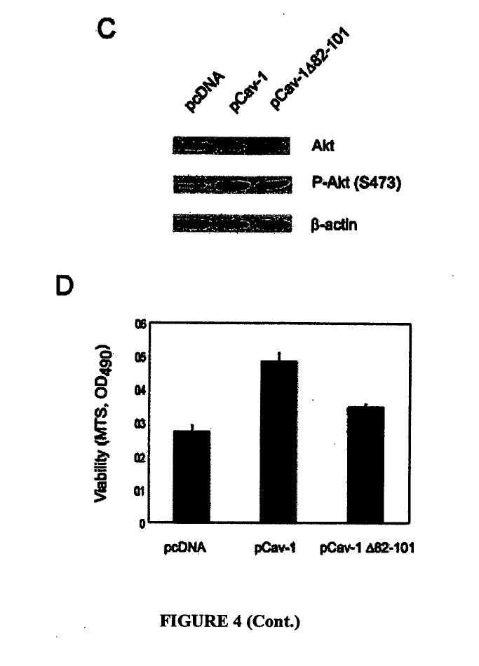

To test the interaction of cav-1 with PP1 and PP2A through a scaffold domain binding site-mediated mechanism, a vector expressing mutated cav-1 with the scaffolding domain deleted (pcav-1Δ82-101) was constructed and used in both IP experiments and serine/threonine protein phosphatase assays. The IP experiments demonstrate that wild-type cav-1 coimmunoprecipitated with PP1 or PP2A, but cav-1 Δ82-101 failed to coimmunoprecipitate with either PP1 or PP2A (FIG. 4A). The results of serine/threonine protein phosphatase assays showed that the deletion of scaffolding domain in cav-1 largely abolished the inhibition of PP1 and PP2A by cav-1 (FIG. 4B: relative activity was determined by measuring the absorbance at 620 nm (A620)). Inserted blots in FIG. 4B show expression of PP1 and PP2A in the same cell lysates. These results indicate that the scaffolding domain in cav-1 is required for its interaction with and inhibition of PP1 and PP2A.

To determine whether deletion of the cav-1 scaffolding domain has an impact on Akt phosphorylation and cav-1-mediated cell protection against Tg-induced apoptosis, Akt phosphorylation analysis and viability assays were performed in LNCaP cells expressing wild-type cav-1 or cav-1Δ82-101 deletion mutant. The results show that deletion of the cav-1 scaffolding domain significantly reduced the level of phosphorylated Akt compared with wild-type cav-1 (FIG. 4C). The results of viability assays also indicate that the scaffolding domain plays an important role in the cav-1-mediated cell protection against Tg-mediated apoptosis (FIG. 4D: after treatment with Tg for 48 h viability was determined in an MTS assay measuring the optical density at 490 nm (OD490) and empty vector pcDNA was used as a control).

### Example 6

### Akt Activity is Largely Responsible for cav-1-Mediated Survival Activities

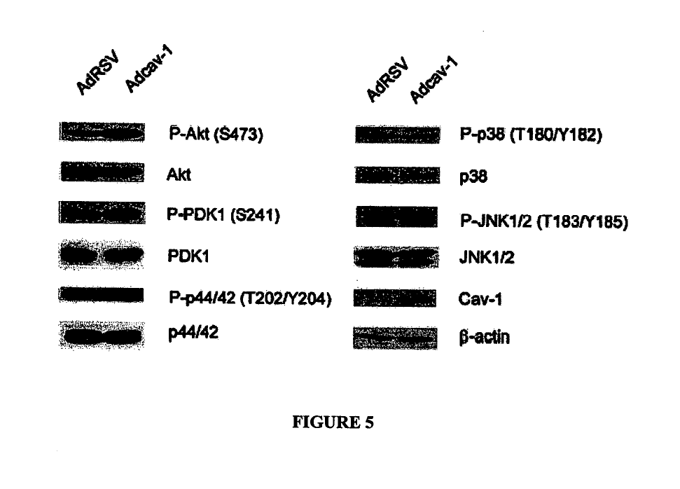

Since PP1 and PP2A have a broad range of substrates and may target different serine/threonine protein kinases, a panel of substrate kinases were examined which are involved in survival pathways, including PDK1, Akt, ERK1/2, p38 MAP kinase, and JNK1/2, in Tg-treated cav-1-expressing cells or vector control cells. The results showed that the expression of cav-1 resulted in higher levels of phosphorylated Akt, PDK1, and ERK1/2; a slightly lower level of phosphorylated p38 MAP kinase; and an unchanged level of phosphorylated JNK1/2 (FIG. 5). Since PDK1 and Akt are in the same pathway, the effect of cav-1 expression on the activity of p38 was minimal, and there was no effect of cav-1 expression on JNK1/2 detected, it was then decided to focus on functional analysis of the Akt and ERK1/2 pathways.

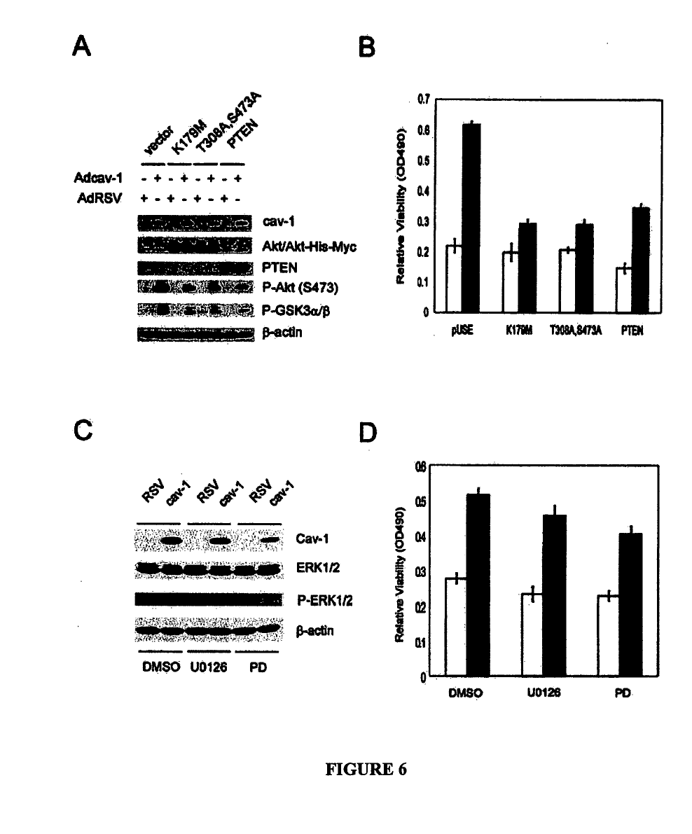

To determine the contribution of Akt pathway cav-1 mediated cell survival, cav-1-expressing cells (Adcav-1) and control cells (AdRSV) were transfected with the following constructs: (i) a control plasmid vector; (ii) a dominant-negative Akt construct carrying a point mutation in its ATP binding pocket (DN-Akt K179M); (iii) a dominant-negative Akt construct bearing defective phosphorylation sites at both T308 and S473 (DN-Akt T308A S473A); (iv) a plasmid vector expressing wild-type PTEN. The data showed that DN-Akt and PTEN proteins were highly expressed in transfected cells, which led to moderate reduction of endogenous active Akt (phosphorylated Akt) and Akt activity (indicated by phosphorylation of downstream target GSK3α/β) (FIG. 6A). The results of viability assays (FIG. 6B: determined by MTS staining followed by measuring the optical density at 490 nm (OD490); cells were infected with AdRSV (£) or Adcav-1 (¢)) showed that in the vector-transfected group the cell viability in cav-1-expressing cells was more than twofold higher in AdRSV-infected cells 48 h after treatment with Tg. Notably, this cav-1-mediated cell protection was largely eliminated in DN-Akt-transfected cells. Interestingly, although cell viability was lower in PTEN-transfected cells relative to the vector-transfected group, the pattern of cell protection by cav-1 remained. These data indicate that Akt activity is largely responsible for cav-1-mediated survival activities and that the action of cav-1 is likely downstream of PTEN.

To evaluate the contribution of ERK1/2 pathway, Adcav-1- or AdRSV-infected cells were treated with U0126, an inhibitor specific to MEK1/2 (direct upstream kinase of ERK1/2) or with PD989059, an inhibitor specific to MEK (MAP kinase kinase). The data in FIG. 6C shows that both U0126 and PD98059 led to reduction of phosphorylated ERK1/2. The results of the viability assay showed that cell viability was reduced by treatment with U0126 and PD98059 about 15 and 20%, respectively, but the reduction was observed in both Adcav-1 and AdRSV-infected cells (FIG. 6D: relative viability determined by measuring the optical density at 490 nm (OD490)). These data indicate that the ERK1/2 pathway may contribute to general cell survival activities appears to be minimal.

### Example 7

### PP1 and PP2A Dephosphorylate Akt In vitro

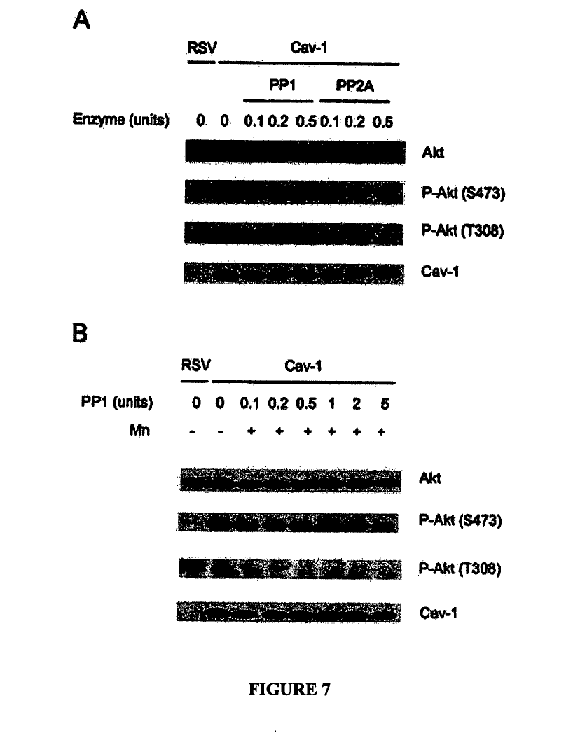

The results above demonstrate that cav-1 maintains phosphoryl ated Akt through interaction with and inhibition of serine/threonine protein phosphatases PP1 and PP2A and that elevated Akt activities are largely responsible for cav-1-mediated survival activities. However, the roles of PP1 and PP2A in the regulation of the Akt pathway in prostate cancer are poorly understood. To address this question, lysates from cav-1-expressing, LY294002-treated cells were incubated with different concentrations of purified PP1 or PP2A enzyme in a protein phosphatase assay buffer (Upstate Biotechnology) or a PP1-specific assay buffer (with 1 mM MnCl2 New England BioLabs) for 30 min at 30° C. The phosphorylation status of Akt in PP1— or PP2A-supplemented cell lysates was compared with control cell lysates from cav-1-expressing or RSV-infected cells. The results presented in FIG. 7 reveal that PP2A effectively dephosphorylates Akt at both S473 and T308 in cav-1-expressing cells in phosphatase assay buffers (without MnCl₂) while PP1 is significantly less effective (FIG. 7A). Interestingly in the presence of MnCl2, PP1 efficiently dephosphorylates Akt at T308 (FIG. 7B). The data indicates that both PP1 and PP2A may regulate the phosphorylation status of Akt. However, PP2A appears to be predominantly Akt phosphatase, since it dephosphorylates Akt at both S473 and T308 efficiently and the activity of PP1 is limited to only T308 and is strictly dependent on Mn2+.

### Example 8

### cav-1 Maintains PDK1 and Akt in a Phosphorylated State

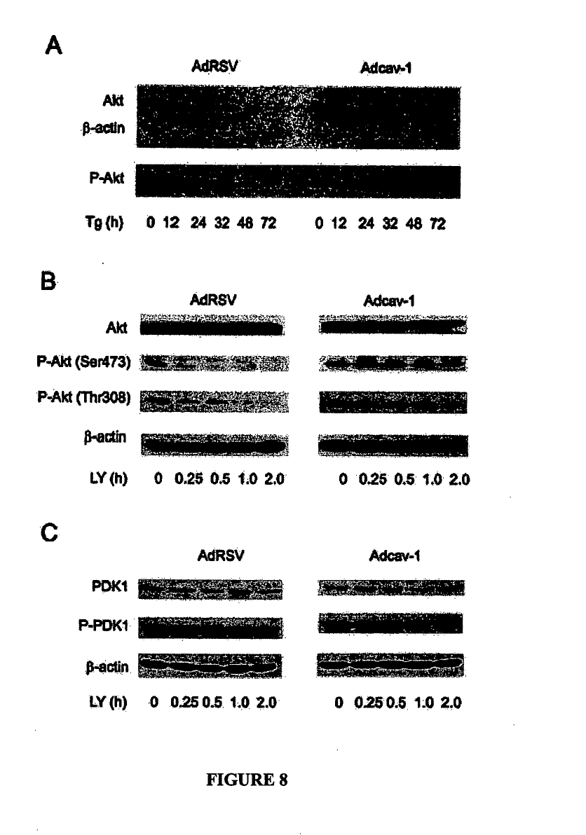

The viability studies described above demonstrated that Akt activity was important in cav-1-mediated cell survival and that enhanced Akt activity was not derived from increased PI3-K activity but instead was potentially derived from the inhibition of serine/threonine protein phosphatases PP1 and PP2A. It was further hypothesized that cav-1 affects Akt protein stability or alters the steady state of phosphorylated Akt in Tg-treated cells. To examine this hypothesis, a time course experiment was performed for the analysis of total Akt and phospho-Akt after treating cav-1-expressing or RSV control LNCaP cells (48 h after infection) with Tg. The experimental results demonstrated that although expression of cav-1 had only a small effect on Akt total protein degradation, it maintained phosphorylated Akt at higher levels over a period of time in Tg-treated cells (FIG. 8A). According to the data analysis by fitting a line to the data points, the half-life of phosphorylated Akt under these experimental conditions in control cell lysates is 65.2 h, whereas in cav-1-expressing cells, it is 224.7 h (Table 1).

TABLE 1
Half-lives of phosphorylation of PDK1 and Akt after the treatment
with PI3-K inhibitor LY294002 or Tg

| Adeno- | Half-life (h) of phosphorylated protein after treatment with: | | | |
|---|---|---|---|---|
| viral | 1 μM Tag | 20 μM LY294002 | | |
| vector | P-Akt (S473) | P-PDK1 (S241) | P-Akt (S473) | P-Akt (T308) |
| Adcav-1 | 224.7 | 13.98 | 2.99 | 2.75 |
| AdRSV | 65.2 | 7.43 | 0.46 | 0.99 |

αThe levels of phosphorylated protein (phosphorylated Akt [P-Akt] or PDK1 [P-PDK1] in FIG. 7 were determined by densitometry (GelExpert; NucleoTech) and normalized to the level of β-actin in the same sample. The half-life was derived from equations generated by fitting a line to the data points.

To confirm the ability of cav-1 to maintain phosphorylated Akt, Akt phosphorylation was analyzed after treating cav-1-expressing or vector control LNCaP cells (48 h after infection with PI3-K-specific inhibitor LY294002). The results presented in FIG. 8B and Table 1 show that the half-life of phosphorylated Akt at S473 in vector control cells is 0.46 h after treatment with inhibitor, whereas the half-life in cav-1-expressing cells was 2.99 h, representing an increase in the half-life of approximately sixfold. The levels of phosphorylation of Akt at T308 were initially higher (approximately twofold) in cav-1-expressing cells relative to that in vector control cells, and its half-life was 2.75 h compared with a half-life of 0.99 h in vector control cells, indicating an approximately threefold increase in the half-life in cav-1-expressing cells.

To evaluate the contribution of upstream kinase PDK1 to the phosphorylation of Akt, the same time course experiment for phosphorylated PDK1 was performed using the same cell lysates (FIG. 8C). Initially, the levels of phosphorylation of PDK1 at S241 were slightly higher (˜17%) in cav-1-expressing cells than in vector control cells. The half-life of phosphorylation of PDK1 in cav-1-expressing cells was 13.98 h, while that in vector control cells was 7.43 h, suggesting an approximately twofold increase in the half-life in cav-1-expressing cells (FIG. 8C and Table 1). These data demonstrate that cav-1 is can maintain phosphorylated Akt after apoptotic challenge or experimental inhibition of Akt phosphorylation. Higher PDK1 activity derived from both a higher initial level and a prolonged half-life of phosphorylated PDK1 may contribute in part to the increase in the half-life of Akt in cav-1-expressing cells.

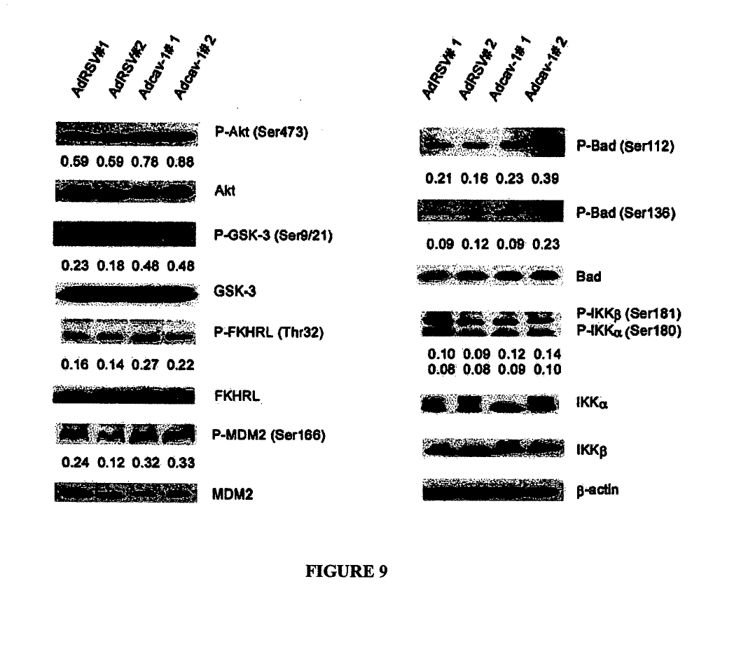

Expression of cav-1 increases phosphorylation of multiple substrates of Akt. Since the Akt pathway is largely responsible for cav-1-mediated cell survival activities in this model system, the phosphorylation state of a panel of Akt substrates were evaluated after the expression of cav-1 and treatment with Tg. The results in FIG. 9 demonstrated that the expression of cav-1 significantly increased the phosphorylation of GSK3 (Ser9/21) (134%), FKHRL1 (Thr32) (47 to 80%), and MDM2 (Ser166) (78 to 81%) and marginally increased phosphorylation of BAD (Ser112) and IKKα (Ser180)/IKKβ (Ser181) but did not significantly alter phosphorylation of CREB (Ser133) and AFX (Ser193) under these conditions (data not shown). Briefly, the phosphorylation state of Akt substrates was analyzed using Tg-treated cav-1-expressing and control cells. Data used were from two independent viral infection experiments (indicated as #1 and #2). The numbers under each gel band are the ratios of phosphorylated protein (indicated by P-prefix before the protein name) to β-actin in the same sample. The protein expression of corresponding molecules in the same cell lysates are also shown. These data clearly demonstrate that the expression of cav-1 leads to significantly higher levels of phosphorylation of specific Akt substrates, which in turn can promote cell survival.

### Example 9

### cav-1 Expression Leads to Increased Nuclear Translocation of AR

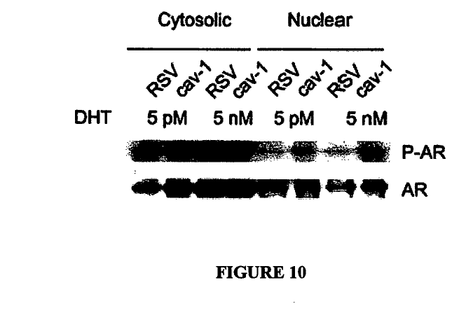

AR has been one of the major focuses in studies of normal and abnormal prostate growth. Although the general molecular mechanism of androgen action has been established, the discovery of specific molecular pathways which lead to precise regulation of AR in either androgen-dependent or -independent manners remains an area of intensive investigation for prostate and prostate malignancies. Because of the potential link of cav-1 with AR activities, AR phosphorylation and its nuclear translocation in cav-1-expressing LNCaP cells was examined. The results from the in vivo phosphorylation of AR demonstrated that the expression of cav-1 in cav-1-negative LNCaP cells led to significantly increased nuclear translocation of phosphorylated AR at both trace (5 pM) and physiological (5 nM) androgen concentrations (FIG. 10), indicating a potential synergetic role for cav-1 in the regulation of AR nuclear translocation. Briefly, forty-eight hours after infection with Adcav-1 or AdRSV, cells were incubated for 6 h in phosphate-free serum-free RPMI 1640 medium containing 5 pM or 5 nM DHT and 200 μCi of ³²P per ml, followed by fractionation of cytosol and nuclear fractions. The top blot shows the effect of cav-1 expression on phosphorylation of AR and nuclear translocation of P-AR in vivo by autoradiography, and the bottom blot shows Western blot analysis of total AR in the same sample. These data together with the data showing increased phosphorylation of Akt substrates GSK3α/β, FKHRL1, and MDM2 strongly demonstrate that cav-1 is able to generate a broad range of cell survival activities.

### Example 10

### cav-1 Expression Leads to Increased Testosterone Uptake

Because of the link between cav-1 expression and AR phosphorylation and nuclear translocation, the effect of cav-1 expression on testosterone uptake was investigated. Briefly, Adcav-1 or AdRSV infected LNCaP cells were incubated for various time periods with radioactively labeled testosterone at a concentration of approximately 0.8 nM. The labeled testosterone media was then washed from the cells and the amount of uptake by the cell measured. The results of this experiment showed that cav-1 expression caused a two-fold increase in the uptake of testosterone after an approximately 2 or 4 h incubation (data not shown). Similar results were obtained in LNCaP cells transfected with a cav-1 expression vector (1-fold increase in testosterone uptake: data not shown). This data indicates that cav-1 overexpression can sensitize cells to increase testosterone uptake thereby providing a mechanism explaining androgen refractory prostate cancer reoccurrence. In other words, the refractory cells are still androgen sensitive but are also capable of taking up bioactive quantities of testosterone even in the presence of concentrations of testosterone consistent with those that are achieved after androgen ablation therapy.

1) A method of identifying agents that modulate the uptake of steroids or steroid-like substances comprising:
a) providing a cell expressing a steroid interacting molecule;
b) contacting the cell with a labeled steroid or steroid-like substance in the presence and absence of at least one agent; and
c) measuring the quantity of steroid or steroid-like substance bound and/or internalized by the cell, wherein a difference in the amount of the steroid or steroid-like substance bound and/or internalized by the cell in the presence of the agent as compared to in its absence is indicative of a modulating agent.

2) The method of claim 1 further comprising a wash step between steps b) and c).

3) The method of claim 1, wherein the steroid interacting molecule being expressed by the cell is exogenous cav-1.

4) The method of claim 3, wherein the cell is stably transfected with an expression vector including the cav-1 gene.

5) The method of claim 1, wherein the cell is of a type selected from the group consisting of: prostate cancer, breast cancer, colon cancer, bladder cancer, squamous cell cancer, papillary carcinoma of the thyroid, ovarian cancers, myeloma, pancreatic ductal adenocarcinoma, renal cancer and lung cancer.

6) The method of claim 5, wherein the cell is an LNCaP cell.

7) The method of claim 1, where in the testosterone is radiolabeled.

8) The method of claim 1, where in the testosterone is fluorescently labeled.

9) The method of claim 1, where in the steroid or steroid-like substance is selected form the group consisting of testosterone, androsterone, progesterone, and progestin.

10) The method of claim 1, where in the steroid interacting molecule is a member of the steroid receptor superfamily.

11) A method for screening and identifying therapeutic agents for the treatment and prevention of neoplastic disorders comprising:
a) expressing a steroid interacting molecule in a cell;
b) contacting the cell with a steroid or steroid-like substance in the presence and absence of at least one agent being screened; and
c) measuring the difference in the uptake of the steroid or steroid-like substance by the cell in the presence and absence of the at least one agent, wherein a change in the amount of steroid or steroid-like substance uptake by the cell in the presence of the at least one agent as opposed to its absence is indicative of a therapeutic agent.

12) A method for screening and identifying therapeutic agents for the treatment and prevention of steroid sensitive neoplastic disorders comprising:
a) expressing a steroid interacting molecule in a cell, wherein the cell is a type of steroid sensitive neoplasia;
b) contacting the cell with a labeled steroid or steroid-like substance in the presence and absence of at least one agent; and
c) measuring the difference in steroid or steroid-like substance binding or internalization by the cell in the presence and absence of the at least one agent, wherein a decrease in the amount of steroid or steroid-like substance binding or internalized by the cell in the presence of the at least one agent as compared to its absence is indicative of a therapeutic agent.

13) A method for screening and identifying therapeutic agents for the treatment and prevention of steroid sensitive neoplastic disorders comprising:
a) expressing a steroid interacting molecule in a cell, wherein the cell is an steroid sensitive neoplasia;
b) contacting the cell with a labeled steroid or steroid-like substance in the presence and absence of at least one agent; and
c) measuring the difference in steroid or steroid-like substance binding or internalization by the cell in the presence and absence of the at least one agent, wherein a decrease in the amount of steroid or steroid-like substance binding or internalized by the cell in the presence of the at least one agent is indicative of a therapeutic agent.

14) A method for screening and identifying therapeutic agents for the treatment and prevention of steroid sensitive neoplastic disorders comprising:
a) providing a cell which expresses a steroid interacting molecule and contains an AR polypeptide;
b) contacting the cell with a steroid or steroid-like substance in the presence and absence of at least one agent; and
c) analyzing the phosphorylation of the AR polypeptide, wherein a difference in the amount of AR polypeptide phosphorylated in the cell in the presence of the agent as compared to in its absence is indicative of a therapeutic agent.

15) An in vivo method of screening and analyzing therapeutic agents for the treatment and prevention of neoplastic disorders comprising:
a) applying a quantity of steroid interacting molecule expressing cells to a defined location in a host animal;
b) applying a quantity of cells lacking expression of the steroid interacting molecule to a different defined location in the host animal;
c) administering an agent to the host animal;
d) administering a steroid or steroid-like substance to the host animal; and
e) analyzing differences in cellular reactions between the cells expressing the steroid interacting molecule and the cells lacking expression of the steroid interacting molecule, wherein differences in the cellular reactions are indicative of a therapeutic agent.

16) A method for screening and identifying therapeutic agents for the treatment and prevention of neoplastic disorders comprising:
a) expressing a cav-1 in a cell;
b) contacting the cell with testosterone in the presence and absence of at least one agent being screened; and
c) measuring the difference in the uptake of the testosterone by the cell in the presence and absence of the at least one agent, wherein a change in the amount of testosterone uptake by the cell in the presence of the at least one agent as opposed to its absence is indicative of a therapeutic agent.
